(19) United States
(12) Patent Application Publication
(10) Pub. No.: US 2021/0315829 A1
(43) Pub. Date: Oct. 14, 2021

(54) Invention Relating to Nanoparticles Containing Taxanes for Administration by Inhalation

(71) Applicant: Justus-Liebig-Universität Gießen, Gießen (DE)

(72) Inventors: Alexandra DALLA-BONA, Heuchelheim (DE); Dr. Tobias GESSLER, Wettenberg (DE); Dr. Savai RAJKUMAR, Bad Nauheim (DE); Dr. Soni SAVAI PULLAMSETTI, Bad Nauheim (DE); Prof. Dr. Ralph SCHERMULY, Mengerskirchen (DE); Prof. Dr. Werner SEEGER, Biebertal (DE)

(21) Appl. No.: 16/645,288

(22) PCT Filed: Sep. 4, 2018
(86) PCT No.: PCT/EP2018/073725

§ 371 Date: Mar. 6, 2020

(30) Foreign Application Priority Data
Sep. 7, 2017 (EP) 17189860.4

(51) Int. Cl. A61K 9/51; A61K 45/06; A61K 9/00; A61K 31/337

(52) U.S. Cl. CPC A61K 9/5138; A61K 45/06; A61K 9/5153; B82Y 5/00; A61K 9/0078; A61K 31/337; A61K 9/5192

(57) ABSTRACT

Nanoparticles comprising at least three polymers, selected independently of one another from the list comprising PLGA, PLA, TPGS, TPGS-750M, PVA, LAEOLA/PLA-PEO-PLA, and also at least one active ingredient, selected from the list comprising paclitaxel, docetaxel and SB-T-1214. The particles have a core-shell structure, the core containing the at least one active ingredient and at least two polymers, the polymers being selected independently of one another from the list comprising RG502H, RG504, PLA, LAEOLA/PLA-PEO-PLA, TPGS and TPGS-750M, the shell containing PVA and/or TPGS. The nanoparticles are nebulizable.

### CROSS REFERENCE TO RELATED APPLICATIONS

This application is a filing under 35 U.S.C. 371 of International Application No. PCT/EP2018/073725 filed Sep. 4, 2018, entitled “Invention Relating to Nanoparticles Containing Taxanes for Administration by Inhalation,” which claims priority to European Patent Application No. 17189860.4 filed Sep. 7, 2017, which applications are incorporated by reference herein in their entirety.

### FIELD OF INVENTION

The present invention relates to the fields of internal medicine, pharmacology and nano-technology.

### BACKGROUND

Pulmonary hypertension (PH) is a severe, life-threatening disease of the human pulmonary vascular system that greatly reduces physical resilience. The rise in pulmonary arterial pressure and in vascular resistance with subsequent dysfunction of the right side of the heart results in a greatly reduced life expectancy, in the case of the subtype of pulmonary arterial hypertension (PAH), the average survival time without treatment is only 2.8 years after diagnosis.

In recent years, various vasoactive active ingredients for treating this disease have been authorized, but they do not lead to a cure or lasting improvement in prognosis. The medicaments hitherto available address the fundamental metabolic pathways of pulmonary vascular regulation: the prostacyclin pathway, nitrogen monoxide (NO) pathway and endothelin pathway. From the group of the prostacyclins, what have been authorized are epoprostenol (intravenous) and the stable prostacyclin analogs iloprost (intravenous and inhalational), treprostinil (intravenous, subcutaneous, inhalational) and beraprost (oral), and additionally selexipag as selective agonist of the prostacyclin IP receptor for oral administration. Medicaments from the NO metabolic pathway are the oral phosphodiesterase 5 inhibitors sildenafil and tadalafil. A first active ingredient from the class of the sGC stimulators or sGC activators for the therapy of PH is provided by riociguat. Bosentan, ambrisentan and macitentan are authorized oral drugs from the group of the endothelin receptor antagonists.

New research results show that pulmonary hypertension is associated with proliferative, antiapoptotic and thus tumor-like changes in pulmonary resistance vessels. In this connection, the forkhead box O (FoxO) transcription factors assume a key role in the control of cellular proliferation; in diseased vessels, they are downregulated or inactivated.

It has been proposed that the pharmacological reconstitution of FoxO activity by the known substance paclitaxel leads to a re-remodeling of the pathologically changed pulmonary resistance vessels and thus to a significant improvement in pulmonary hypertension.

Paclitaxel is a known chemotherapeutic from the group of the taxanes and is available as Abraxane® or Taxol® for intravenous treatment of various tumor diseases. However, the paclitaxel excipients added in each case, paclitaxel being only sparingly soluble in water, limit the pulmonary administration of paclitaxel. In the case of Taxol®, the solubilizer macrogolglycerol ricinoleate (cremophor) that is added damages the gas exchange-essential function of the pulmonary surfactant system. In the case of Abraxane , paclitaxel is bound to albumin nanoparticles of an average size of approximately 130 nanometers. In therapy studies with inhalational administration of nebulized Abraxane® in patients with severe PH, what became apparent was an initially very good tolerability of the medicament. However, in the case of repetitive administration of the medicament, what developed was a hypersensitivity syndrome with allergic reactions to the human albumin used in Abraxane®, meaning that therapeutic use of Abraxane® for aerosol therapy of PH is not possible, as has been proposed in, for example, US patent 2014/0271871 A1.

Inhalational administration is the administration of active ingredients in the form of an aerosol via inhalation into the lungs, also called pulmonary administration. It is a suitable way of allowing easy-to-use administration for the patient. The patient is spared undesired systemic adverse effects associated with intravenous or oral administration. Furthermore, the amount of active ingredient used can be distinctly reduced, since, in the case of inhalational administration, the active ingredient is brought directly to the lungs and prior distribution and dilution through the whole body does not take place. Inhalational administration is particularly advantageous in the administration of active ingredients for therapy of lung diseases.

The European Pharmacopeia differentiates between the following preparations suitable for inhalational administration:
preparations which are converted into vapor
liquid preparations for nebulization
liquid preparations in pressurized-gas dose inhalers
powders for inhalation

Instruments named by the European Pharmacopeia that can administer the preparations for inhalation are:
nebulizers
inhalers
pressurized-gas dose inhalers
standard-pressure dose inhalers
powder inhalers

There is a need for a way of formulating paclitaxel and other taxanes such as docetaxel or second-generation taxanes such as SB-T-1214, and of providing a preparation, in such a manner to make them amenable to inhalational administration, for example to be able to administer the active ingredient by inhalation as an aerosol. It would thus be possible for the first time to provide patients with pulmonary hypertension (PH) with active ingredients and therapeutics which promise a better efficacy owing to their antitumor properties. Pulmonary hypertension treatment that is specific and is low in adverse effects is facilitated by the direct inhalational administration of the active ingredient into the lungs.

Numerous materials such as, for example, biocompatible nanoparticles have long been known as suitable active-ingredient carrier systems for so-called drug delivery, including for taxanes. In the case of paclitaxel, nanoparticle formulations for intravenous tumor therapy have been described (e.g., Bernabeu et al.; Paclitaxel: What has been done and the challenges remain ahead, Int J Pharm. 2017 526(1-2):474-495). It is true that these known nanoparticles are suitable for intravenous use. However, they are not stable with respect to nebulization and are therefore not suitable for inhalational administration or for use in a nebulizer.

In the case of the pulmonary administration of other active ingredients such as treprostinil and iloprost, although nanoparticle carrier systems are known, they are, however, not suitable for the use of taxanes such as docetaxel or second-generation taxanes such as SB-T-1214 and especially paclitaxel because they cannot take up the active ingredient, the taxanes, since it is not water-soluble. These carrier systems are not nebulizable and also not stable with respect to the aerosol-generation process, which exhibits a high level of mechanical stress due to pressure application, ultrasound treatment and/or thermal treatment.

Currently, there are no nebulizable taxane formulations based on biocompatible nanoparticles that are suitable for inhalational administration.

What would be desirable would be the direct transport of nanoparticle-encapsulated taxanes such as docetaxel or second-generation taxanes such as SB-T-1214, especially paclitaxel, into the lungs via inhalational administration, with the result that a controlled release of active ingredient at the site of action, the lungs, is achieved in order to allow the treatment of pulmonary hypertension with taxanes such as docetaxel or second-generation taxanes such as SB-T-1214, especially paclitaxel.

### SUMMARY

It is an object of the present disclosure to provide biocompatible nanoparticles for the inhalational administration of active ingredient such as paclitaxel and other taxanes such as docetaxel or second-generation taxanes such as SB-T-1214 that are suitable for inhalational administration and for treatment of pulmonary hypertension. The particles are to be easy-to-produce, have a high degree of loading with active ingredient, and are storage-stable and stable with respect to the processes of aerosol generation, such as, for example, pressure application, ultrasound treatment and/or thermal treatment.

According to the disclosure, this object is achieved by the nanoparticles disclosed herein.

### BRIEF DESCRIPTION OF THE DRAWINGS

FIG. 1 shows the nebulization profile of the particles from exemplary embodiment 1.

FIG. 2 shows the release kinetics of the particles from exemplary embodiment 1.

FIG. 3 shows the nebulization profile of the particles from exemplary embodiment 2.

FIG. 4 shows the release kinetics of the particles from exemplary embodiment 2.

FIG. 5A shows results of the measurement of the proliferation index of PTX-F1.

FIG. 5B shows results of the measurement of the proliferation index of PTX-F2.

FIG. 6A shows results of the measurement of the apoptosis index of PTX-F1.

FIG. 6B shows results of the measurement of the apoptosis index of PTX-F2.

FIG. 7A shows the release kinetics of PTX-F1/PTX-F2 of up to 4 hours.

FIG. 7B shows the release kinetics of PTX-F1/PTX-F2 of up to 12 hours.

FIG. 7C shows the release kinetics of PTX-F1/PTX-F2 of up to 550 hours.

FIG. 8 shows the measurement results of the stability tests on the basis of preparations PTX-F1 and PTX-F2 that were performed by way of example.

FIG. 9 shows a table of the produced particles according to exemplary embodiments 1-12 including preparations PTX-F1 and PTX-F2.

### DETAILED DESCRIPTION

Surprisingly, it was possible to provide biocompatible nanoparticles containing the active ingredient paclitaxel or else other taxanes such as docetaxel or second-generation taxanes such as SB-T-1214 that are nebulizable as aerosol and are thus very highly suited to inhalational administration.

Advantageously, the particles have a high degree of loading with active ingredient and are easy-to-produce. They are stable with respect to the process of aerosol generation, i.e., pressure application, ultrasound treatment and/or thermal treatment. The storage stability of the particles becomes apparent in the physicochemical properties, such as size, zeta potential and loading, not having substantially changed after 11 months. A storage stability of at least 11 months can be demonstrated.

The nanoparticles are produced from the biodegradable polymers PLGA (poly(lactide-co-glycolide)), PLA (polylactide), PLA-PEO-PLA (poly(lactide-b-ethylene oxide-b-lactide)) together with an alpha-tocopherol derivative, TPGS (D-alpha-tocopherol polyethylene glycol 1000 succinate) or TPGS-750-M (and DL-α-tocopherol methoxypolyethylene glycol succinate), and PVA (poly(vinyl alcohol)) in various compositions by means of a modified solvent evaporation method. The result is PVA-coated, active ingredient-containing biocompatible nanoparticles. In one embodiment, paclitaxel-containing nanoparticles are present. In further embodiments, docetaxel-containing nanoparticles or second-generation taxane (SB-T-1214)-containing nanoparticles.

In particular, the nanoparticles comprise at least three polymers, selected independently of one another from the list comprising PLGA, PLA, TPGS, TPGS-750M, PVA, LAEO-LA/PLA-PEO-PLA, and also at least one active ingredient, selected from the list comprising paclitaxel, docetaxel and SB-T-1214.

The particles have a core-shell structure, the core containing the at least one active ingredient and at least two polymers and said polymers being selected independently of one another from the list comprising RG502H, RG504, PLA, LAEOLA/PLA-PEO-PLA, TPGS and TPGS-750M. The shell contains PVA and/or TPGS.

These PVA-coated, active ingredient-containing nanoparticles have a high active ingredient-loading efficiency, based on the total particle weight, between 1% and 80%, preferably between 3% and 70%, particularly preferably between 5% and 50%, very particularly preferably between 10% and 40%. The size is 200-3000 nm.

After production, the nanoparticles are present as a nanoparticle suspension. Said nano-particle suspension is nebulized as an aerosol by means of a conventional aerosol generator, for example a piezoelectric aerosol generator, with the physicochemical properties of the nanoparticles remaining unchanged. The particles exhibit a composition-dependent release of active ingredient, as shown exemplarily for paclitaxel in in vitro experiments. Furthermore, a therapeutic effect becomes apparent in pulmonary-hypertension cell cultures (human IPAH-PASMC cells). The nanoparticles and the nanoparticle suspension produced therefrom thus prove to be suitable for nebulization and thus for inhalational administration of paclitaxel, docetaxel, second-generation taxane (SB-T-1214). They are a suitable therapeutic for treating pulmonary hypertension.

A further aspect of the disclosure is a method for producing the nanoparticles.

The nanoparticles are hybrid matrix nanoparticles that include a combination of biocompatible polymers, active ingredient (e.g., selected from paclitaxel, docetaxel, second-generation taxanes such as SB-T-1214) and a tocopherol derivative in their matrix and a protective PVA coat. The active ingredient is an extremely complex active ingredient, since it is only sparingly soluble and decomposes rapidly in aqueous media. Production from an aqueous medium is therefore not possible. With regard to the solvent evaporation method, there is additionally the limitation that the use of polar organic solvents (ethanol, acetone) leads to hardening of the active ingredient in its crystalline form (acicular crystals): since ethanol and acetone are water-soluble, these two solvents immediately dissolve in the resultant aqueous mixture, the result being that the active ingredient precipitates, specifically in a highly undefined crystalline form, the biological availability of which is not adjustable. Although this also occurs more slowly, possibly even only incompletely, in the case of polar organic solvents which are only partially or little soluble in water, it nevertheless leads to undefined, pharmacologically unusable particles. It is thus important to use nonpolar organic solvents such as, for example, dichloromethane in order to maintain the active ingredient in its amorphous structure. PVA, as the coat, and the tocopherol derivative react synergistically in this case in order to prevent the active ingredient from crystallizing out. The PVA coating additionally protects the particles from the influence of the kinetic energy of nebulization and allows a long storage stability as an aqueous suspension, and this is advantageous with respect to nebulization in vibrating mesh nebulizers (only liquids are nebulizable). A poloxamer coating is not suitable because of the high water solubility and the incompatibility with the tissue of the lungs.

A further aspect is the need for a reliable buffer for setting a neutral pH of 7.4. Polymers based on lactic acid or glycolic acid tend to split autocatalytically into the contained monomers under slightly acidic to acidic conditions. Thus, a constantly neutral pH must prevail during production in order to preserve the conformity of the polymers. Surprisingly, HEPES was found to be a suitable buffer. HEPES is a short name known to a person skilled in the art and stands for 2-(4-(2-hydroxyethyl)-1-piperazinyl)ethane sulfonic acid. HEPES is distinguished by a good tolerability for humans, since it does not diffuse through cell membranes and therefore, inter alia, does not bear a hazardous-substance label (GHS). Furthermore, HEPES as a buffer substance barely interacts with polymers. It exhibits high solubility and is very cost-effective to produce.

The polymers PLA and PLGA that are used are, when used individually, unsuitable for encapsulating the active ingredient in a sufficient manner, since no suitable particle properties can be generated. Only the combination of PLA and PLGA leads to very good particle properties and to a very good loading.

Furthermore, the desired release profile can be achieved by means of the mixing ratio. Here, carrier substance and stabilizer in the matrix are PLGA (in different monomer ratios and functional end groups) and TPGS or TPGS 750-M. By introducing hydrophilic components such as PLA or triblock copolymers such as PLA-PEO-PLA, it is possible to create porous structures, through which the active ingredient can be released in a purposeful manner (sustained release).

Finally, it is alternatively possible to clean up and subsequently filter the nanoparticle suspension in order to obtain nebulizable particle suspensions. Firstly, this can ensure the removal of organic solvent residues, and, secondly, PVA residues and other polymer residues, which could possibly impair nebulization due to clogging of nebulizer pores, are removed.

### General Instructions for Producing Nanoparticles According to the Disclosure

The nanoparticles are produced in a method that is easy to carry out and comprises the following steps:

a) preparing an aqueous phase comprising 0.01% to 1%, preferably 0.05%, of a polymer selected from the list comprising PVA and TPGS and/or the buffer substance HEPES, involving dissolving the polymer and/or the buffer substance HEPES with stirring and adjusting the pH to a value between 7 and 8, preferably to about 7.4, and cooling the aqueous phase to 19° C. to 25° C., preferably to 22° C. Alternatively, the aqueous phase is additionally filtered after preparation.

b) preparing an organic phase comprising at least two polymers selected from the list comprising RG502H, RG504, LAEOLA, PLA, TPGS and TPGS-750M, the concentration of the at least two polymers being 10 mg/ml to 30 mg/ml, preferably about 15 mg/ml, at least one active ingredient selected from the list comprising paclitaxel, docetaxel and SB-T-1214, the concentration of the at least one active ingredient being 5 mg/ml to 15 mg/ml, preferably about 10 mg/ml, involving dissolving the at least two polymers and the at least one active ingredient in a nonpolar aprotic organic solvent selected from the list comprising methylene chloride and ethyl acetate. Alternatively, the aqueous phase is additionally filtered after preparation.

c) mixing 5 parts of the aqueous phase from step a) with one part of the organic phase from step b) to yield an emulsion which is finely emulsified at 0° C. to 30° C., preferably at 0° C. to 4° C., over a period of 1 to 5 minutes, preferably about 3 minutes, using a high-performance disperser at 10 000 revolutions/min to 30 000 revolutions/min, preferably at about 21 000 revolutions/min, so that the particle size of the emulsion is further reduced. Alternatively, the emulsion is yielded at a temperature of 19-22° C., with constant stirring at a stirring speed between 300 revolutions/min and 700 revolutions/min, preferably about 500 revolutions/min.

d) homogenizing the emulsion from step c) using an ultrasonic homogenizer at 0° C. to 30° C., preferably at 0° C. to 4° C. Alternatively, the homogenization is carried out initially for 0.5 to 2 minutes, preferably 1 minute, with a power of about 25 watts and then for 0.5 to 2 minutes, preferably 1 minute, with a power of about 30 watts. In a further alternative, the homogenization is carried out for 2 to 5 minutes, preferably 3 minutes, with a power of 20 to 40 watts, preferably with a power of 25 to 30 watts, or with an amplitude of 20% to 90%, preferably with an amplitude of 30% to 75%, particularly preferably with an amplitude of 60% to 75%. Alternatively, the ultrasonic homogenization is carried out with or without pulsation, the ratio between “duration of break” and “duration of sonic action” for the pulsation being within the range from 1:20 to 2:1, preferably about 1:2.

e) preparing the particle suspension by extracting the organic solvent from the homogenisate from step d) under reduced pressure at 20° C. to 50° C., preferably at 35° C., over a period of 0.5 h to 5 h, preferably over a period of 2 h to 3 h.

Alternatively, clean-up of the particle suspension is carried out in an additional step f) after step e) by centrifuging the particle suspension from step e) one to five times, preferably three times, over a period of 20 to 50 minutes, preferably about 30 minutes, at 0° C. to 30° C., preferably at about 4° C., with an acceleration of 10 000 g to 20 000 g, preferably with an acceleration of about 14 000 g, the supernatant being replaced with fresh purified water each time and the particles being resuspended in between by means of ultrasound in an ultrasonic bath.

Alternatively, a further additional step g) is carried out after step e) or after step f) for filtration of the particle suspension through a filter having a pore size between 2 μm and 8 μm, preferably of about 5 μm.

In an example of use on the laboratory scale, what is carried out in parallel batches is the rapid injection of, in each case, 1 ml of organic phase into, in each case, 5 ml of aqueous phase at room temperature using a Hamilton syringe and with constant stirring (500 rpm). The resultant emulsions are pooled and further emulsified, on ice (max. 4° C.) or uncooled, for 3 minutes (min) using a high-performance disperser (Ultraturrax®, IKA®-Labortechnik) at 21 000 revolutions/min. The emulsion thus obtained is further refined using a tip sonicator (Sonopuls®, Bandelin) for 3 min, likewise on ice (max. 4° C.) or uncooled, at an amplitude of 30-60% (alternatively for 1 min at 25 watts and for 1 min at 30 watts) with or without pulsation, i.e., the introduction of the ultrasonic power with or without interruptions of the flow of waves. For example, the following pulse sequences can be used: 3 minutes at 25-30 watts or an amplitude of 60-75%, and at an interval of, in each case, 0.5 s break and 1 s sonic action.

The solvent fraction (organic phase) is subsequently evaporated in a rotary evaporator under reduced pressure at 35° C. for approx. 2-3 h. Here, the emulsion gives rise to a suspension. The suspended particles are centrifuged three times (14 000 g, 30 min, 4° C.). The supernatant is replaced with fresh purified water each time and the particles are resuspended by means of ultrasound in an ultrasonic bath. Finally, the particle suspension is filtered through a 5μm nylon push-on filter. The suspension can be stored for up to 11 months, and the pure particles have a shelf life of distinctly over 11 months.

Paclitaxel, docetaxel or second-generation taxanes such as SB-T-1214 are commercially available, for example from Abmole Bioscience. Poly(lactide-co-glycolide), PLGA for short, is commercially available, for example as Resomer RG502H (acid terminated, Mw 7000-17 000) or Resomer® RG504 (lactide:glycolide 50:50, ester terminated, Mw 38 000-54 000) and Resomer® RG504H (acid terminated, lactide:glycolide 50:50, Mw 38 000-54 000) from Boehringer Ingelheim. Poly(vinyl alcohol), PVA for short, is commercially available, for example as Mowiol® 4-88. D-alpha-Tocopherol polyethylene glycol 1000 succinate, TPGS for short, and DL-α-tocopherol methoxypolyethylene glycol succinate, TPGS-750-M for short, is commercially available, for example from Sigma Aldrich. Polylactide, PLA for short, is commercially available, for example from Polysciences. Poly(lactide-b-ethylene oxide-b-lactide), PLA-PEO-PLA or LAEOLA for short, (LA:EO:LA ratio corresponds to 2:8:2) and polylactide, PLA for short, (15 kD) is commercially available, for example from Polysciences. HEPES is commercially available, for example from Sigma Aldrich.

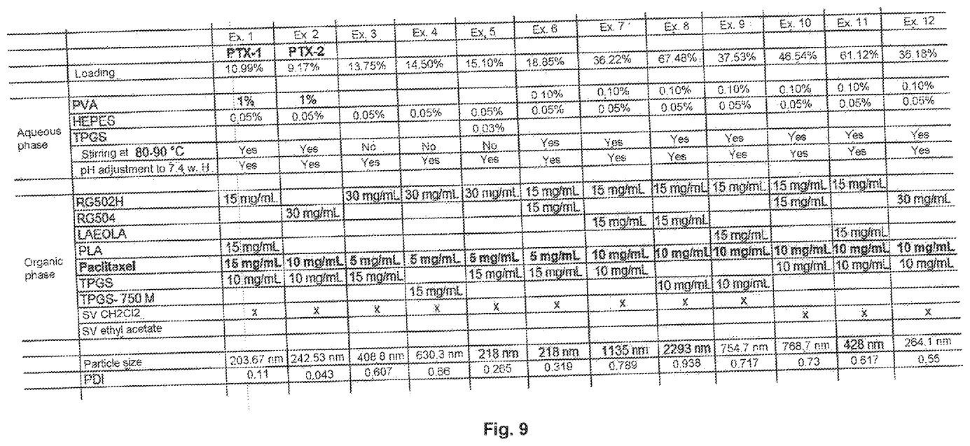

By way of example, 12 exemplary embodiments relating to the particles are disclosed, the particle composition of which is clearly presented in the table in FIG. 9.

### Characterization of the Nanoparticles

After the production process, the particle suspension containing the active ingredient paclitaxel or other taxanes such as docetaxel or second-generation taxanes such as SB-T-1214 is measured and characterized as follows with respect to size and the PDI (dispersity index or, previously, polydispersity index). To this end, 10 μl of the particle suspension containing approx. 10 mg/ml nanoparticles are removed after clean-up and taken up in 1 ml of purified water. In this diluted dispersion, the particles are then measured with respect to size and PDI. Particle mass and active-ingredient loading are determined by drying 3×1 ml particle suspension, undiluted, overnight at 60° C. in weighed-out vessels, for example from Eppendorf, and determining the yield gravimetrically. Recovery, loading and loading capacity are deduced from the determination of concentration by means of HPLC. Loading capacity, i.e., the maximum achievable loading, based on the amount of active ingredient used, is consistently never below 90%.

### Experimental Description of Nebulizability/Particle Size

The particle suspension containing the active ingredient paclitaxel or one of the other taxanes such as docetaxel or second-generation taxanes such as SB-T-1214 are nebulized. The nebulization is carried out using a nebulizer as known in the prior art, for example eFlow® (PARD, with addition of 1 mM CaCl2 as electrolyte. The reference used is 1 mM CaCl2 solution.

The mass median aerodynamic diameter (MMAD) of the aerosol drops is ascertained by means of laser diffraction (HELOS, Sympatec). All the measurements are analyzed in Mie mode. The geometric standard deviation (GSD) is calculated as follows by means of the measured laser diffraction values:

GSD=√(d_(84%/d_(16%))

d_(n) is the drop diameter at the percentage n of the relevant size distribution.

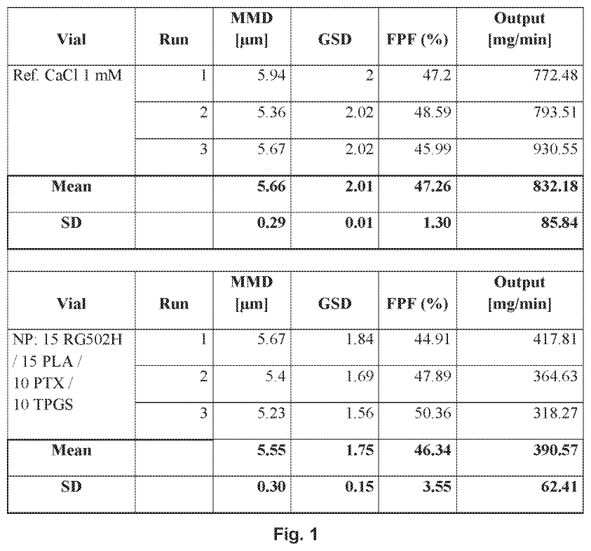

The “fine particle fraction” (FPF) corresponds to the volume amount of aerosol having drop sizes less than 5.25 μm. The output (aerosol mass flow), i.e., the nebulized aerosol mass from the nebulizer per minute, is determined by weighing the nebulizer before and after the nebulization process (cf FIG. 1 and FIG. 3).

### Experimental Description of Release Kinetics

2 ml of nanoparticle suspension containing the active ingredient paclitaxel or one of the other taxanes such as docetaxel or second-generation taxanes such as SB-T-1214 is embedded are centrifuged in an undiluted state, the clear supernatant is discarded and the residue is topped up to 2 ml with saline phosphate buffer (PBS, pH 7.4, 0.1% sodium dodecyl sulfate).

The samples thus obtained are incubated at 37° C. with constant shaking. At predetermined time points, 100 μl are removed each time and centrifuged, and the pellet is analyzed for the content of active ingredient remaining by means of HPLC. The concentration values in the pellets that are thus obtained are used to mathematically ascertain what is currently the concentration in the supernatant (“free PTX”).

After the production process, the polymeric nanoparticles in which the active ingredient, for example paclitaxel or else other taxanes such as docetaxel or second-generation taxanes such as SB-T-1214, is embedded are tested with respect to the effects of especially their cytotoxicity, proliferation and apoptosis on human IPAH-PASMC cells. These are smooth muscle cells from human pulmonary arteries (PASMC), which were isolated from lungs from patients with idio-pathic pulmonary arterial hypertension (IPAH). By way of example, the two particle suspensions PTX-F1 and PTX-F2 were used in the in vitro tests.

### a) Determination of Cytotoxicity

By way of example, the two particle suspensions PTX-F1 and PTX-F2 were tested with respect to their cytotoxicity at varying concentration and for different lengths of time on human IPAH-PASMC cells. In this case, it became apparent that both do not have a toxic effect on the cells. Said cells exhibited unchanged vitality and no discernible morphological changes or death.

### b) Determination of Proliferation

The in vitro determinations of the effects of the particle suspensions PTX-F1 and PTX-F2 that were carried out by way of example clearly show the antiproliferative effect on human IPAH-PASMC cells.

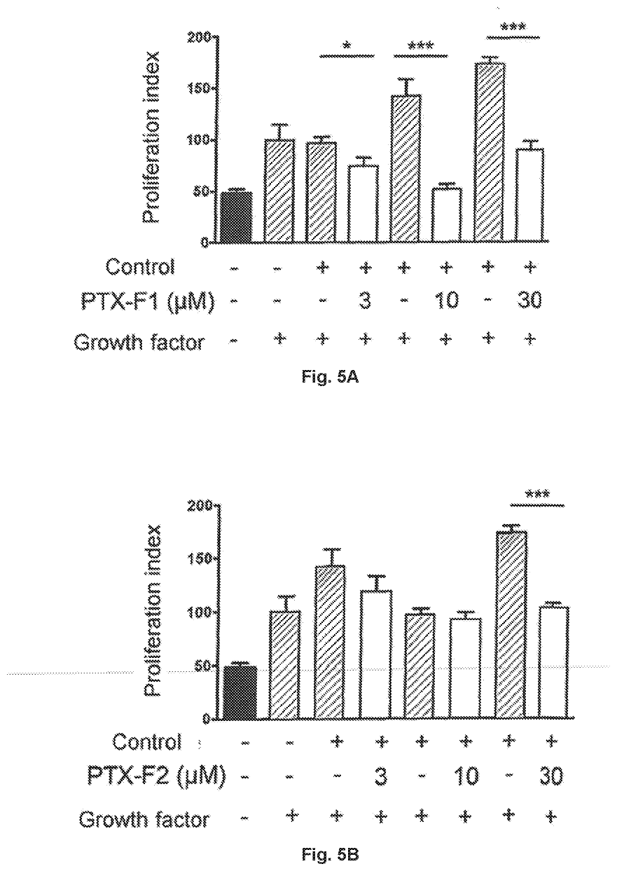

Particle suspension PTX-F1 significantly reduced the growth factor-stimulated proliferation of human IPAH-PASMC cells in a dose-dependent manner (FIG. 5A). PTX-F2, by contrast, likewise exhibited antiproliferative effects, though at higher concentrations (FIG. 5B). Nanoparticles without active ingredient, which were produced according to an above-mentioned method with no addition of active ingredient in step b), were used as a control.

After serum withdrawal for 48 hours, the human IPAH-PASMC cells were stimulated with 5% fetal calf serum (FCS). Thereafter, the cells were incubated with particle suspension PTX-F1 or PTX-F2 in concentrations of 3, 10 and 30 μM for 24 hours with and without dimethyl sulfoxide (DMSO) as carrier. Thereafter, proliferation was measured by means of the bromodeoxyuridine (BrDU) proliferation assay. For the statistical evaluation, the values of the test groups (PTX-F1 or PTX-F2) were compared with the values of the DMSO carrier group in Student's t-test. The significance levels were defined at *=p<0.05 and ***=p<0.001.

### c) Determination of Apoptosis

By way of example, the two particle suspensions PTX-F1 and PTX-F2 were tested with respect to their apoptotic effect on human IPAH-PASMC cells.

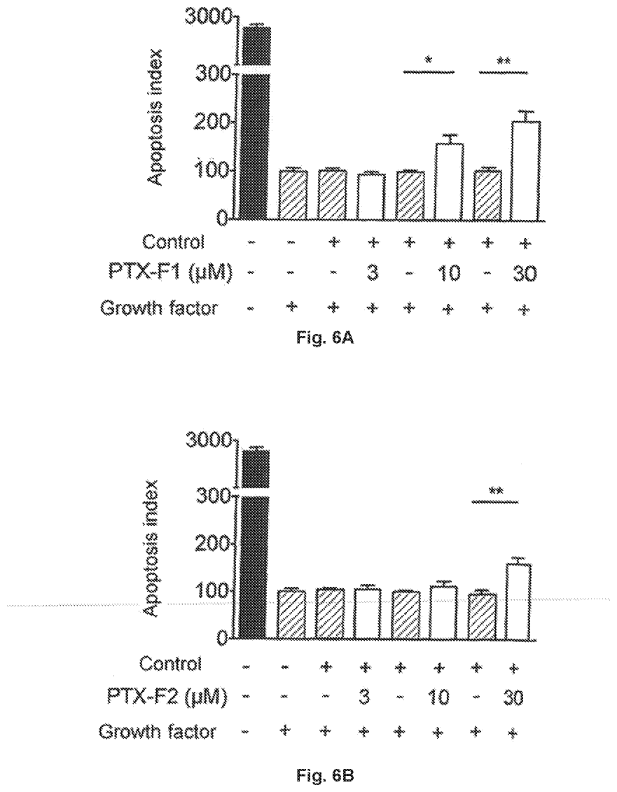

Stimulation with growth factors brings about a strong resistance of the IPAH-PASMC cells to apoptosis in comparison with nonstimulated IPAH-PASMC cells. Particle suspension PTX-F1 significantly increased the growth factor-induced apoptosis resistance of human IPAH-PASMC cells in a dose-dependent manner (FIG. 6A). PTX-F2, by contrast, exhibited a growth factor-induced apoptosis resistance only at high concentrations (FIG. 6B). Nanoparticles without active ingredient were used as a control.

After serum withdrawal for 48 hours, the human IPAH-PASMC cells were stimulated with 5% fetal calf serum (FCS). Thereafter, the cells were incubated with PTX-F1 or PTX-F2 in concentrations of 3, 10 and 30 μM for 24 hours with and without dimethyl sulfoxide (DMSO) as carrier. Thereafter, apoptosis was measured by means of the TUNEL apoptosis assay. For the statistical evaluation, the values of the test groups (PTX-F1 or PTX-F2) were compared with the values of the DMSO carrier group in Student's t-test. The significance levels were defined at *=p<0.05 and ***=p<0.001.

### d) Determination of Release Kinetics

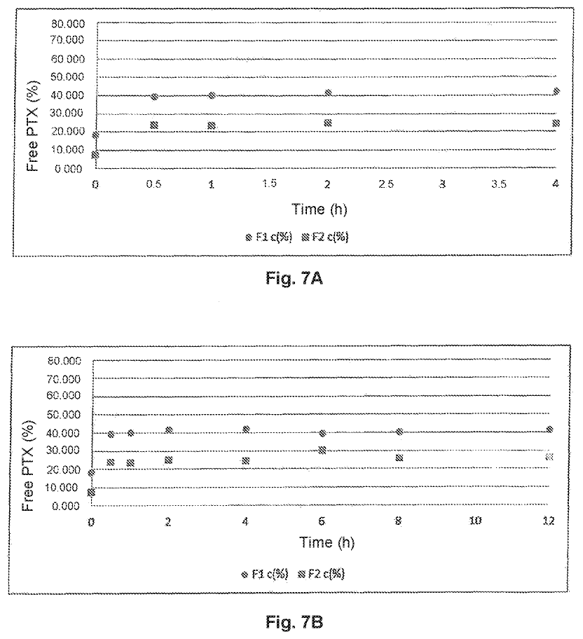

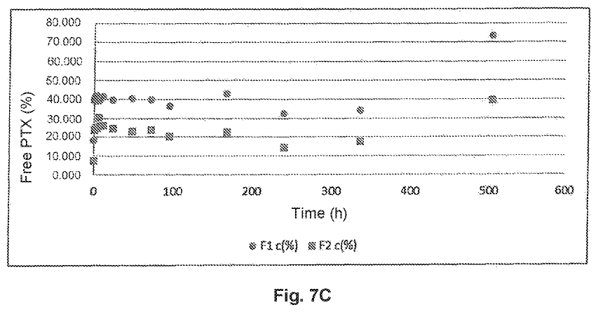

The exemplary determination of release kinetics according to the experimental procedure disclosed further above on the basis of paclitaxel yielded the following result (cf FIGS. 7A, 7B and 7C).

The nanoparticles according to the invention are distinguished by having a dynamic release kinetics for the contained active ingredient. Said dynamic release kinetics comprise at least two temporally successive phases: the temporally first phase is a so-called “burst phase”, i.e., a period in which a particularly rapid release of active ingredient takes place (duration: up to about 2 hours, but preferably shorter, for example about 1 h), meaning that a substantially constant concentration of the active ingredient in the liquid phase outside the particles is achieved after just a short time. The achievement of this substantially constant concentration marks the end of the burst phase that is distinct in each case for each distinct particle variant according to the invention. What then directly temporally follows the burst phase is the plateau phase, in which a slower release of the active ingredient takes place, the result being that the concentration in the liquid phase that was achieved in the burst phase continues to be substantially maintained. Said plateau phase extends over a period of up to about 4 hours, preferably over a period of up to about 12 hours, further preferably over a period of up to about 150 hours, and very particularly preferably over a period of up to about 400 hours (FIGS. 7A, 7B and 7C). Because of these release kinetics, it is possible with the nanoparticles according to the invention to release at least one active ingredient, for example paclitaxel or else other taxanes such as docetaxel or second-generation taxanes such as SB-T-1214, both in a rapid manner and in a sustained manner (depot effect), and this is extremely advantageous for a treatment.

### e) Determination of Storage Stability

Storage stability was ascertained by using the parameters size, zeta potential and active-ingredient content. A person skilled in the art is well aware of the methods and procedures from the prior art that are suitable for this purpose.

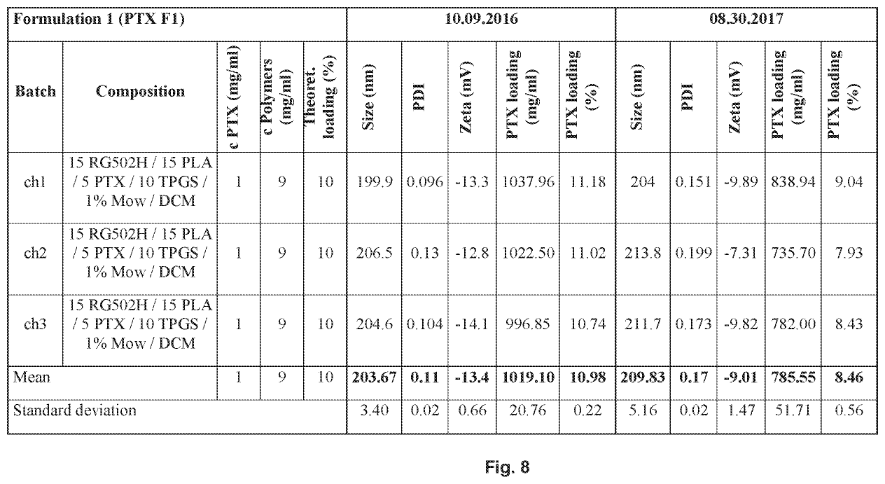

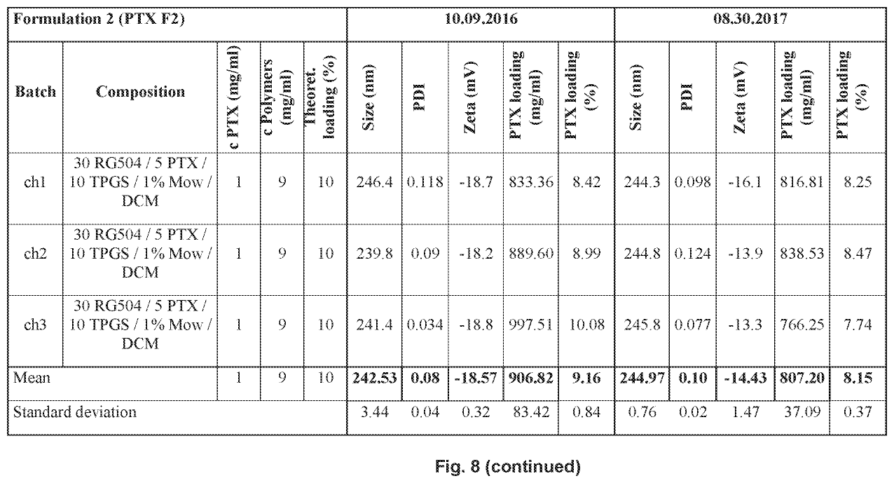

By way of example, storage stability is determined on the basis of paclitaxel-containing nanoparticles according to the invention. The results are shown in FIG. 8: after 11 months of storage in distilled water at 0-4° C., the particle sizes, the PDI and the zeta potential are substantially unchanged; the percentage loading with active ingredient (paclitaxel in the example) is somewhat lowered in both examples (11% vs. 8.5% and 9.2% vs. 8.2%), but still exceptionally good. The particles according to the invention have a very good storage stability.

### EXAMPLES

### Exemplary Embodiment 1

This example (also referred to as PTX-1) shows PLGA-PLA-TPGS-PVA nanoparticles in which the active ingredient paclitaxel is embedded. Loading is 10.99%. Aqueous phase: 1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HC;, and the solution is filtered after cooling. Organic phase: 15 mg/ml RG502H, 15 mg/ml PLA, 10 mg/ml paclitaxel and 10 mg/ml TPGS are dissolved in dichloromethane and the solution is filtered. In the case of these nanoparticles, the shell contains the polymer PVA and the core contains the polymers RG502H, PLA and TPGS and also paclitaxel as active ingredient. The polymers in the core are present in relation to one another approximately in the ratio of 3:3:2 (RG502H:PLA:TPGS). The content of paclitaxel, based on the total particle mass, is approximately 10% (percent by weight). The particle size is 203.67 nm and the PDI is 0.11. Production is carried out as described above.

FIG. 1 shows the nebulization profile of exemplary embodiment 1.

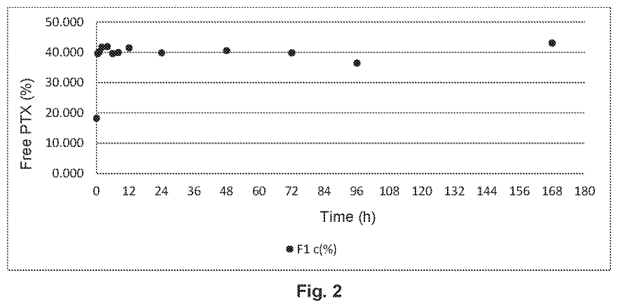

FIG. 2 shows the release kinetics of exemplary embodiment 1.

### Exemplary Embodiment 2

This example (also referred to as PTX-2) shows PLGA-PLA-TPGS-PVA nanoparticles in which the active ingredient paclitaxel is embedded. Loading is 9.17%. Aqueous phase: 1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 30 mg/ml RG504, 10 mg/ml paclitaxel and 10 mg/ml TPGS are dissolved in dichloromethane and the solution is filtered. In the case of these nanoparticles, the shell contains the polymer PVA and the core contains the polymers RG504 and TPGS and paclitaxel as active ingredient. The polymers in the core are present in relation to one another approximately in the ratio of 3:1 (RG504:TPGS). The content of paclitaxel, based on the total particle mass, is approximately 10% (percent by weight). The particle size is 242.53 nm and the PDI is 0.043. Production is carried out as described above.

FIG. 3 shows the nebulization profile of exemplary embodiment 2.

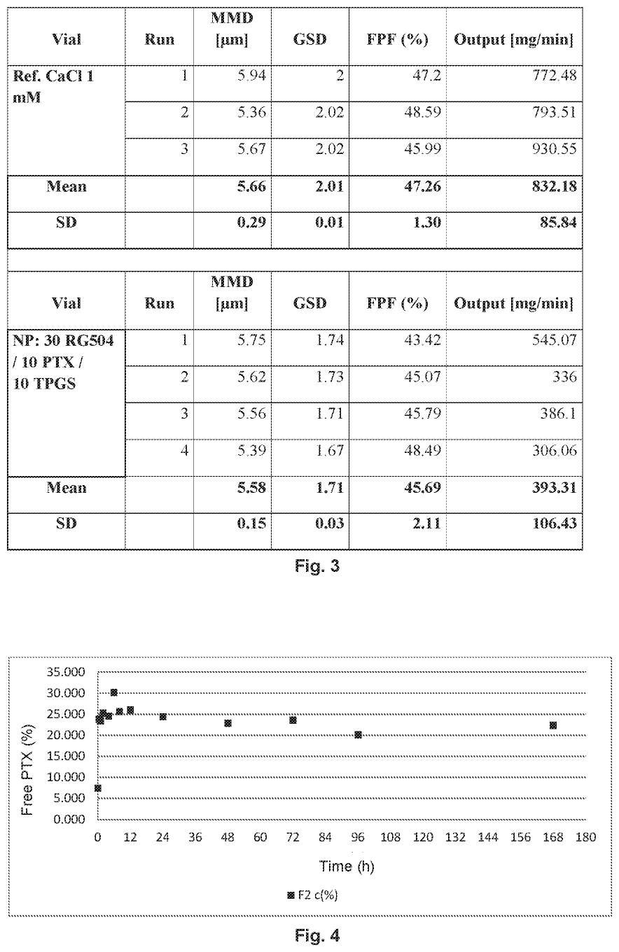

FIG. 4 shows the release kinetics of exemplary embodiment 2.

### Exemplary Embodiment 3

This example shows PLGA-TPGS nanoparticles in which the active ingredient paclitaxel is embedded. Loading is 13.75%. Aqueous phase: 0.05% HEPES (2-(4-(2-hydroxyethyl)-1-piperazinyl)ethanesulfonic acid) is dissolved with constant stirring, the pH is adjusted to 7.4 using HCl and the solution is filtered. Organic phase: 30 mg/ml RG502H, 5 mg/ml paclitaxel and 15 mg/ml TPGS are dissolved in dichloromethane and the solution is filtered. The particle size is 408.8 nm and the PDI is 0.607. Production is carried out as described above.

### Exemplary Embodiment 4

This example shows PLGA-TPGS nanoparticles in which the active ingredient paclitaxel is embedded. Loading is 14.5%. Aqueous phase: 0.05% HEPES (2-(4-(2-hydroxyethyl)-1-piperazinyl)ethanesulfonic acid) is dissolved with constant stirring, the pH is adjusted to 7.4 using HCl and the solution is filtered. Organic phase: 30 mg/ml RG502H, 5 mg/ml paclitaxel and 15 mg/ml TPGS-750 M are dissolved in dichloromethane and the solution is filtered. The particle size is 630.3 nm and the PDI is 0.66. Production is carried out as described above.

### Exemplary Embodiment 5

This example shows PLGA-TPGS-TPGS nanoparticles in which the active ingredient paclitaxel is embedded. Loading is 15.10%. Aqueous phase: 0.03% TPGS and 0.05% HEPES (2-(4-(2-hydroxyethyl)-1-piperazinyl)ethanesulfonic acid) are dissolved with constant stirring, the pH is adjusted to 7.4 using HC1 and the solution is filtered. Organic phase: 30 mg/ml RG502H, 5 mg/ml paclitaxel and 15 mg/ml TPGS are dissolved in dichloromethane and the solution is filtered. The particle size is 218 nm and the PDI is 0.265. Production is carried out as described above.

### Exemplary Embodiment 6

This example shows PLGA-PLGA-TPGS-PVA nanoparticles in which the active ingredient paclitaxel is embedded. Loading is 18.85%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 15 mg RG504, 15 mg RG502H, 5 mg/ml paclitaxel and 15 mg/ml TPGS are dissolved in dichloromethane and the solution is filtered. The particle size is 218 nm and the PDI is 0.319. Production is carried out as described above.

### Exemplary Embodiment 7

This example shows PLGA-LAEOLA-TPGS-PVA nanoparticles in which the active ingredient paclitaxel is embedded. Loading is 36.22%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 15 mg/ml RG502H, 15 mg/ml LAEOLA, 10 mg/ml paclitaxel and 10 mg/ml TPGS are dissolved in dichloromethane and the solution is filtered. The particle size is 1135 nm and the PDI is 0.789. Production is carried out as described above.

### Exemplary Embodiment 8

This example shows PLGA-LAEOLA-TPGS-PVA nanoparticles in which the active ingredient paclitaxel is embedded. Loading is 67.48%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 15 mg/ml RG502H, 15 mg/ml LAEOLA, 10 mg/ml paclitaxel and 10 mg/ml TPGS-750-M are dissolved in dichloromethane and the solution is filtered. The particle size is 2293 nm and the PDI is 0.938. Production is carried out as described above.

### Exemplary Embodiment 9

This example shows PLGA-PLA-TPGS-PVA nanoparticles in which the active ingredient paclitaxel is embedded. Loading is 37.53%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 15 mg/ml RG502H, 15 mg/ml PLA, 10 mg/ml paclitaxel and 10 mg/ml TPGS-750-M are dissolved in dichloromethane and the solution is filtered. The particle size is 754.7 nm and the PDI is 0.717. Production is carried out as described above.

### Exemplary Embodiment 10

This example shows PLGA-PLGA-TPGS-PVA nanoparticles in which the active ingredient paclitaxel is embedded. Loading is 46.54%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 15 mg/ml RG504, 15 mg/ml RG502H, 10 mg/ml paclitaxel and 10 mg/ml TPGS are dissolved in ethyl acetate and the solution is filtered. The particle size is 768.7 nm and the PDI is 0.73. Production is carried out as described above.

### Exemplary Embodiment 11

This example shows PLGA-PLA-TPGS-PVA nanoparticles in which the active ingredient paclitaxel is embedded. Loading is 61.12%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HC1, and the solution is filtered after cooling. Organic phase: 15 mg/ml RG502H, 15 mg/ml PLA, 10 mg/ml paclitaxel and 10 mg/ml TPGS are dissolved in ethyl acetate and the solution is filtered. The particle size is 428 nm and the PDI is 0.617. Production is carried out as described above.

### Exemplary Embodiment 12

This example shows PLGA-TPGS-PVA nanoparticles in which the active ingredient paclitaxel is embedded. Loading is 36.18%. Aqueous phase: 0.1% PVA and 0.05% HEPES (2-(4-(2-hydroxyethyl)-1-piperazinyl)ethanesulfonic acid) are dissolved at 80-90° C. with constant stirring, the pH is adjusted to 7.4 using HCl, and the solution is filtered after cooling. Organic phase: 30 mg/ml RG504, 10 mg/ml paclitaxel and 10 mg/ml TPGS are dissolved in ethyl acetate and the solution is filtered. The particle size is 264.1 nm and the PDI is 0.55. Production is carried out as described above.

What is claimed is:

1. Nanoparticles comprising at least three polymers, selected independently of one another from the list comprising PLGA, PLA, TPGS, TPGS-750M, PVA, LAEOLA/PLA-PEO-PLA, and also at least one active ingredient, selected from the list comprising paclitaxel, docetaxel and SB-T-1214, wherein
i) the particles have a core-shell structure, the core containing the at least one active ingredient and at least two polymers, the polymers being selected independently of one another from the list comprising RG502H, RG504, PLA, LAEOLA/PLA-PEO-PLA, TPGS and TPGS-750M, and
ii) the shell contains PVA and/or TPGS, and
iii) the nanoparticles are nebulizable.

2. The nanoparticles as claimed in claim 1, wherein the particles have dynamic release kinetics for the at least one active ingredient, said dynamic release kinetics comprising at least two temporally successive phases, a rapid release taking place in a first initial burst phase of up to about 2 hours, the result being that a substantially constant concentration in the liquid phase outside the particles is achieved up to the end of the burst phase, and a slow release taking place in a plateau phase which temporally follows the burst phase and which extends over a period of up to about 4 hours, the result being that the concentration in the liquid phase that was achieved in the burst phase is substantially maintained.

3. The nanoparticles as claimed in claim 1, wherein loading with the at least one active ingredient, based on the total particle weight, is between 1% and 80%.

4. The nanoparticles as claimed in claim 1, wherein the shell contains the polymer PVA and the core contains the polymers RG502H, PLA and TPGS and also paclitaxel as medicinal active ingredient, the polymers in the core being present in relation to one another approximately in the ratio of 3:3:2 (RG502H:PLA:TPGS) and the content of paclitaxel, based on the total particle mass, being approximately 10% (percent by weight).

5. The nanoparticles as claimed in claim 1, wherein the shell contains the polymer PVA and the core contains the polymers RG504 and TPGS and also paclitaxel as medicinal active ingredient, the polymers in the core being present in relation to one another approximately in the ratio of 3:1 (RG504:TPGS) and the content of paclitaxel, based on the total particle mass, being approximately 10% (percent by weight).

6. The nanoparticles as claimed in claim 1, having a storage stability of up to 11 months in dispersion and/or over 11 months in substance/pure form, the storage stability of the particles presenting itself in a substantially constant size of the nanoparticles and a substantially constant zeta potential of the nanoparticles and a decrease in the active-ingredient concentration of not more than 30%.

7. A method for producing the nanoparticles as claimed in claim 1, comprising the steps of:
a) preparing an aqueous phase comprising 0.01% to 1%, of a polymer selected from the list comprising PVA and TPGS and/or the buffer substance HEPES, involving dissolving the polymer and/or the buffer substance HEPES with stirring and adjusting the pH to a value between 7 and 8, and cooling the aqueous phase to 19° C. to 25° C.
b) preparing an organic phase comprising
at least two polymers selected from the list comprising RG502H, RG504, LAEOLA, PLA, TPGS and TPGS-750M, the concentration of the at least two polymers being 10 mg/ml to 30 mg/ml,
at least one active ingredient selected from the list comprising paclitaxel, docetaxel and SB-T-1214, the concentration of the at least one active ingredient being 5 mg/ml to 15 mg/ml,
involving dissolving the at least two polymers and the at least one active ingredient in a nonpolar aprotic organic solvent selected from the list comprising methylene chloride and ethyl acetate.
c) mixing 5 parts of the aqueous phase from step a) with one part of the organic phase from step b) to yield an emulsion which is further emulsified at 0° C. to 30° C., over a period of 1 to 5 minutes, using a high-performance disperser at 10 000 revolutions/min to 30 000 revolutions/min, so that the particle size is reduced.
d) homogenizing the emulsion from step c) using an ultrasonic homogenizer at 0° C. to 30° C.,
e) preparing the particle suspension by extracting the organic solvent from the homogenisate from step d) under reduced pressure at 20° C. to 50° C., over a period of 0.5 h to 5 h.

8. The method for producing the nanoparticles as claimed in claim 7, wherein, in step c), the mixing of the aqueous phase according to step a) with the organic phase according to step b) is carried out at a temperature of 19-22° C. and with constant stirring at a stirring speed between 300 revolutions/min and 700 revolutions/min.

9. The method for producing the nanoparticles as claimed in claim 7, wherein, in step d), the homogenization of the emulsion from step c) is carried out over a first period of 0.5 to 2 minutes, with a power of about 25 watts and then over a second period of 0.5 to 2 minutes, with a power of about 30 watts.

10. The method for producing the nanoparticles as claimed in claim 7, wherein, in step d), the homogenization of the emulsion from step c) is carried out over a period of 2 to 5 minutes, with a power of 20 to 40 watts or with an amplitude of 20% to 90%.

11. The method for producing the nanoparticles as claimed in claim 7, wherein, in step d), the homogenization of the emulsion from step c) is carried out with or without pulsation, the ratio between “duration of break” and “duration of sonic action” for the pulsation being within the range from 1:20 to 2:1.

12. The method for producing the nanoparticles as claimed in claim 7, wherein a further step f) is carried out after step e) for clean-up of the particle suspension by centrifuging the particle suspension from step e) one to five times over a period of 20 to 50 minutes at a temperature of 0° C. to 30° C. with an acceleration of 10 000 g to 20 000 g, the supernatant being replaced with fresh purified water each time and the particles being resuspended in between by ultrasound in an ultrasonic bath.

13. The method for producing the nanoparticles as claimed in claim 7, wherein a further step g) is carried out after step e) or after step f) for filtration through a filter having a pore size between 2 μm and 8 μm.

14. The use of the nanoparticles as claimed in claim 1 for inhalational administration of at least one active ingredient selected from the list comprising paclitaxel, docetaxel and SB-T-1214 in a nebulizer.

15. The use of the nanoparticles as claimed in claim 1 for treating pulmonary hypertension.
